(19) United States
(12) Patent Application Publication
(10) Pub. No.: US 2018/0116975 A1
(43) Pub. Date: May 3, 2018

(54) COMPOSITIONS AND COMBINATIONS FOR THE TREATMENT OF ANGIOGENESIS DISEASES AND DISORDERS

(71) Applicant: BIOMAS LTD., Jerusalem (IL)

(72) Inventor: Benjamin SREDNI, Kfar Saba (IL)

(73) Assignee: FERAMDA LTD., Kfar-Saba (IL)

(21) Appl. No.: 15/553,184

(22) PCT Filed: Feb. 25, 2016
(86) PCT No.: PCT/IL2016/050213

§ 371 Date: Aug. 24, 2017

Related U.S. Application Data
(60) Provisional application No. 62/121,225, filed on Feb. 26, 2015.

(51) Int. Cl. A61K 31/095; A61K 9/00; A61P 27/02; C07K 16/22; A61K 39/395; C07K 14/71; A61K 38/17

(52) U.S. Cl. CPC A61K 31/095; A61K 9/0053; A61K 9/0014; A61K 9/0048; A61P 27/02; C07K 2319/30; A61K 39/3955; C07K 14/71; A61K 38/179; C07K 2317/24; C07K 2317/55; C07K 16/22

(57) ABSTRACT

The present invention relates to compositions, combinations and methods for treating, reducing, ameliorating, alleviating, or inhibiting the progression of, angiogenesis diseases and disorders in a patient in need thereof (such as for example AMD), utilizing Te-containing compounds.

### TECHNOLOGICAL FIELD

The present application relates to compositions, combinations and methods for treating, reducing, ameliorating, alleviating, or inhibiting the progression of, angiogenesis diseases and disorders in a patient in need thereof.

### BACKGROUND

Angiogenesis is a vital process in the growth of new capillary blood vessels in the body is an important natural process in the body used for healing and reproduction. The body controls angiogenesis by producing a precise balance of growth and inhibitory factors in healthy tissues. When this balance is disturbed, the result is either too much or too little angiogenesis. Abnormal blood vessel growth either excessive or insufficient is now recognized as a “common denominator” underlying many deadly and debilitating conditions including cancer, skin diseases, age related blindness, diabetic ulcers, cardiovascular disease, stroke and many others. The list of diseases that have angiogenesis as an underlying mechanism grows longer every year.

Angiogenesis is an important natural process occurring in the body both in health and in disease. Angiogenesis occurs in the healthy body for healing wounds and for restoring blood flow to tissues after injury or insult. In females angiogenesis also occurs during the monthly reproductive cycle (to rebuild the uterus lining, to mature the egg during ovulation) and during pregnancy (to build the placenta, the circulation between mother and fetus).

The healthy body controls angiogenesis through a series of “on” and “off” switches: The main “on” switches are known as angiogenesis stimulating growth factors and the “off switches” are known as angiogenesis inhibitors, When angiogenic growth factors are produced in excess of angiogenesis inhibitors the balance is tipped in favor of blood vessel growth. When inhibitors are present in excess of stimulators angiogenesis is stopped. The normal healthy body maintains a perfect balance of angiogenesis modulators. In general angiogenesis is “turned off” by the production of more inhibitors than stimulators.

In many serious diseases states the body loses control over angiogenesis and angiogenesis dependent diseases result when new blood vessels either grow excessively or insufficiently.

Excessive angiogenesis occurs in diseases such as cancer, diabetic blindness, age related macular degeneration, rheumatoid arthritis, psoriasis and other conditions. In these conditions new blood vessels feed diseased tissues, destroy normal tissues and in the case of cancer the new vessels allow tumor cells to escape into the circulation and lodge in other organs (tumor metastases). Excessive angiogenesis occurs when diseased cells produce abnormal amounts of angiogenic growth factors overwhelming the effects of natural angiogenesis inhibitors. Antiangiogenic therapies are aimed to halt new blood vessel growth there by used to treat these conditions.

Insufficient angiogenesis occurs in diseases such as coronary artery disease, stroke and chronic wounds. In these conditions blood vessel growth is inadequate and circulation is not properly restored leading to the risk of tissue death. Insufficient angiogenesis occurs when tissues cannot produce adequate amounts of angiogenic growth factors. Therapeutic angiogenesis is aimed to stimulate new blood vessel growth with growth factors is being developed to treat these conditions.

Angiogenesis dependent therapies restoring the body's natural control of angiogenesis provide a comprehensive approach to fighting disease. By using medical treatments that either inhibit or stimulate angiogenesis, doctors are prolonging the lives of cancer patients, preventing limb amputations, reversing vision loss and improving general health.

Most solid cancerous tumors release angiogenic growth factor proteins that stimulate blood vessels to grow into the tumor, providing it with oxygen and nutrients, Anti-angiogenic therapies literally starve the tumor of its blood supply by interfering with this process. Cancer treatments that block angiogenesis are suitable for treating cancer types including colon cancer, kidney cancer, lung cancer, breast cancer and liver cancer, as well as multiple myeloma and bone gastrointestinal stromal tumors.

Angiostatic therapies designed to inhibit neovascularization associated with multiple pathological conditions have only been partially successful; complete inhibition has not been achieved. There is still a need for angiogenesis therapy that target distinct aspects of the angiogenic process, resulting in better inhibition of neovascular growth associated with development, ischemic retinopathy, and tumor growth, with little or no effect on normal, mature tissue vasculature.

### GENERAL DESCRIPTION

The inventors of the present application have surprisingly found that administering to a subject suffering from a disease or disorder associated with angiogenesis a Te-containing compound as defined herein above in combination with an anti-angiogenesis compound (either prior, during or after the treatment with said Te-containing compound) provides far reaching benefits to the reduction, amelioration, alleviation, or inhibition of the progression of, angiogenesis diseases and disorders or symptoms thereof.

The inventors have found that a combination as disclosed herein below was able to provide beneficial therapeutic effects to a patient that suffers from an angiogenesis condition, disorder or disease that was continuously administered with an angiogenesis inhibitor (the same or different than the inhibitor in the combination of the invention) or that was administered in the past with an angiogenesis inhibitor (the same or different than the inhibitor in the combination of the invention). Such benefits include, but are not limited to, the reduction of administration of said angiogenesis inhibitor to said patient. For example, if said patient has been administered with said angiogenesis inhibitor alone every month for the treatment of said angiogenesis condition, disorder or disease, upon administration of a the Te-containing compound said angiogenesis inhibitor will be administered to said patient less frequently than once a month.

The present invention provides a combination comprising at least one Te-containing compound having the formula I or II

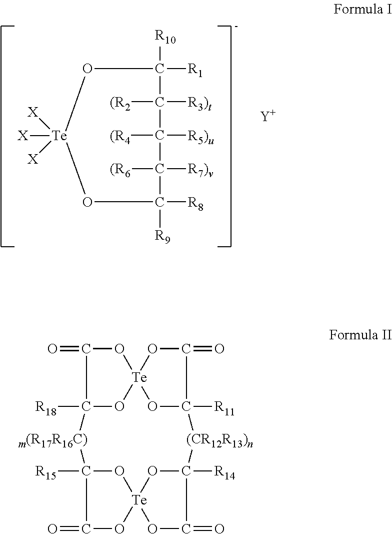

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

and at least one angiogenesis inhibitor.

In some embodiments, said at least one Te-containing compound is a compound of formula I. In other embodiments, said at least one Te-containing compound is a compound of formula II.

In some embodiments, said at least one Te-containing compound is a compound of formula I, wherein t, u and v are each 0 and wherein each of R₁, R₈, R₉ and R₁₀ is hydrogen, wherein X is chloro and wherein Y is ammonium. In some further embodiments, said Te-containing compound is AS-101 below:

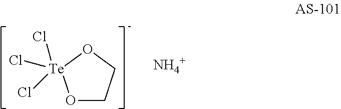

In some embodiments, said at least one Te-containing compound is a compound of formula II, wherein each of m and n is 0 and wherein each of R₁₁, R₁₄, R₁₅ and R₁₈ is hydrogen. In some further embodiments, said Te-containing compound is SAS below:

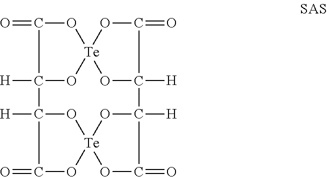

The term “angiogenesis inhibitor” encompasses any substance that inhibits the growth of new blood vessels (angiogenesis). In some embodiments, said angiogenesis inhibitor is from an endogenous source (i.e. part of the body's control). In other embodiments said angiogenesis inhibitor is an exogenous compound (either a synthetic or semi-synthetic drug or a dietary supplement).

In some embodiments, said at least one angiogenesis inhibitor is selected from a group consisting of growth factor inhibitor, VEGF inhibitor, Placental growth factor inhibitor, Fibroblast growth factors (1 and 2) inhibitor, Pleiotrophin inhibitor, Platelet derived growth factor inhibitor, Transforming growth factor-a inhibitor, Transforming growth factor-β inhibitor, Epidermal Growth Factor inhibitor, Insulin-like growth factor inhibitor, Hepatocyte growth factor inhibitor, Angiopoietins inhibitor, Angiopoietin-1 inhibitor, Angiopoietin-2 inhibitor, Cytokines and chemokines, Tumor necrosis factor-α inhibitor, Interleukin-8 and 3 inhibitor, Prostaglandin E1 inhibitor, E2 inhibitor, Enzymes, Thymidine phosphorylase inhibitor, Cycloxygenase-2 inhibitor, Angiogenin inhibitor, Cell adhesion molecules inhibitor, Integrins, Vascular cell adhesion molecule-1 inhibitor, E-Selectin inhibitor, Vascular endothelial cadhedrin inhibitor, Plasminogen activators, Matrix metalloproteinase, Plasminogen activator inhibitor, Oestrogens, Proliferin, Leptin, Erythropoetin, Granulocyte colony stimulating factor inhibitor, Granulocyte-monocyte colony inhibitor and stimulating factor inhibitor, or any combination thereof.

Angiogenesis inhibitors can also be found in natural sources, which include, but are not limited to at least one of: Angioarrestin, Angiostatin (plasminogen fragment), Antiangiogenic antithrombin III, Cartilage-derived inhibitor (CDI), CD59 complement fragment, Endostatin (collagen XVIII fragment), Fibronectin fragment, Gro-beta, Heparinases, Heparin hexasaccharide fragment, Human chorionic gonadotropin (hCG), Interferon alpha/beta/gamma, Interferon inducible protein (IP-10), Interleukin-12, Kringle 5 (plasminogen fragment), Metalloproteinase inhibitors (TIMPs), 2-Methoxyestradiol, Placental ribonuclease inhibitor, Plasminogen activator inhibitor, Platelet factor-4 (PF4), Prolactin 16 kD fragment, Proliferin-related protein (PRP), Retinoids, Tetrahydrocortisol-S, Thrombospondin-1 (TSP-1), Transforming growth factor-beta (TGF-b), Vasculostatin, Vasostatin (Calreticulin fragment) or any combinations thereof.

In some embodiments, said at least one angiogenesis inhibitor is selected from agents for the treatment of cancer, including but are not limited to Bevacizumab (Avastin), Axitinib (Inlyta), Cabozantinib (Cometriq), Pazopanib (Votrient), Regorafenib (Stivarga®), Sorafenib (Nexavar®), Sunitinib (Sutent®), Vandetanib (Caprelsa®), Temsirolimus (Torisel®), Everolimus (Afinitor®), Interferon alfa (Intron® A and Roferon®), Lenalidomide (Revlimid®), Thalidomide (Thalomid®), rhEndostatin (Endostar/Endu) or any combinations thereof; agent for the treatment of eye disease, including but not limited to: Ranibizumab (Lucentis®), Pegaptanib (Macugen®), Aflibercept)(Eylea©) Ozurdex©, Iluvien© or any combinations thereof; agent for dermatological administration or indication, including but not limited to Alitretinoin (Panretin® 0.1% gel, Ligand), Imiquimod (Aldara® 5% cream, Zyclara 3.75% cream, Medicis), Polyphenon E (Veregen® 15% ointment, Bradley/MediGene), or any combinations thereof.

In some further embodiments, said at least one angiogenesis inhibitor active agent is selected from the group consisting of Ranibizumab, Aflibercept, Bevacizumab or any combinations thereof.

In some embodiments, said at least one Te-containing compound is formulated for oral administration. In other embodiments, said at least one Te-containing compound is formulated for topical administration. In further embodiments, said at least one Te-containing compound is formulated for intraocular administration. In other embodiments, said at least one Te-containing compound is formulated for targeted in-situ administration (i.e. administration into the angiogenentic growth. Such administration may be performed via targeting moieties or directly into the growth to be treated. For example directly into a cancerous growth or into an eye of a subject affected with for example AMD). In some embodiments, said at least one Te-containing compound is formulated for parenteral administration.

The present invention also relates to compositions comprising a Te-containing compound in admixture with pharmaceutically acceptable auxiliaries, and optionally other therapeutic agents. The auxiliaries must be “acceptable” in the sense of being compatible with the other ingredients of the composition and not deleterious to the recipients thereof.

Pharmaceutical compositions include those suitable for oral, rectal, nasal, topical (including transdermal, buccal and sublingual), vaginal or parenteral (including subcutaneous, intramuscular, intravenous and intradermal) administration or administration via an implant. The compositions may be prepared by any method well known in the art of pharmacy.

Such methods include the step of bringing in association compounds used in the invention or combinations thereof with any auxiliary agent. The auxiliary agent(s), also named accessory ingredient(s), include those conventional in the art, such as carriers, fillers, binders, diluents, disintegrants, lubricants, colorants, flavoring agents, anti-oxidants, and wetting agents.

Pharmaceutical compositions suitable for oral administration may be presented as discrete dosage units such as pills, tablets, dragees or capsules, or as a powder or granules, or as a solution or suspension. The active ingredient may also be presented as a bolus or paste. The compositions can further be processed into a suppository or enema for rectal administration.

The invention further includes a pharmaceutical composition, as hereinbefore described, in combination with packaging material, including instructions for the use of the composition for a use as hereinbefore described.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated. Compositions or formulations suitable for pulmonary administration e.g. by nasal inhalation include fine dusts or mists which may be generated by means of metered dose pressurized aerosols, nebulisers or insufflators.

In another aspect, the invention provides a combination comprising at least one Te-containing compound having the formula I or II

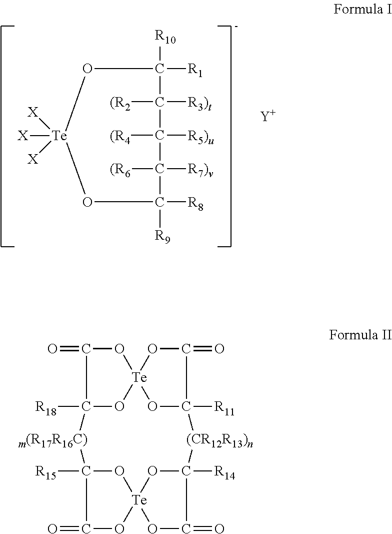

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

and at least one angiogenesis inhibitor, for use in the treatment of angiogenesis conditions, diseases, disorders and symptoms thereof in a subject in need thereof.

When referring to an “angiogenesis condition, disease, disorder and symptom thereof”, it should be understood to encompass any condition, disease, disorder or symptom associated with excessive vascular growth (see Bisht M. et al. Indian J Pharmacol. 2010 42(1): 2-8). Such conditions include, but are not limited to: cancer, arthritis, psoriasis, and blinding retinopathy, inflammatory, allergic, infectious, traumatic, metabolic or hormonal diseases, such as, atherosclerosis, restenosis, transplant arteriopathy, warts, scar keloids, synovitis, osteomyelitis, asthma, nasal polyps, choroideal and intraocular disorders (including macular degeneration), retinopathy of prematurity, diabetic retinopathy, AIDS, endometriosis, and uterine bleeding. Studies have also shown that a high-fat diet induces an angiogenic gene program in fat tissues, which stimulates adipogenesis. Infectious diseases are also angiogenic. Viral and bacterial pathogens carry angiogenic genes or induce the expression of angiogenic genes in the host. The human herpesvirus 8 transforms EC and causes Kaposi's sarcoma in HIV-1 infected AIDS patients. Similar to diabetic retinopathy, there is increased glomerular expression of vascular growth factors that contribute to diabetic nephropathy by promoting vessel leakage.

In some embodiments, said angiogenesis conditions is selected from a group consisting of cancer, arthritis, psoriasis, blinding retinopathy, atherosclerosis, restenosis, transplant arteriopathy, warts, scar keloids, synovitis, osteomyelitis, asthma, nasal polyps, choroideal, intraocular disorders, retinopathy of prematurity, diabetic retinopathy, AIDS, endometriosis, uterine bleeding and obesity or any combinations thereof.

In some further embodiments, said angiogenesis conditions include any disease associated with the proliferation of blood vessels. In some other embodiments, said angiogenesis conditions include cancer, macular degeneration in the eye. In some further embodiments, said angiogenesis condition includes wet age related macular degeneration (wet AMD) and dry age related macular degeneration (dry AMD).

Macular degeneration is the leading cause of severe vision loss in people over age 60. It occurs when the small central portion of the retina, known as the macula, deteriorates. The retina is the light-sensing nerve tissue at the back of the eye. Macular degeneration is a source of significant visual disability.

There are two main types of age-related macular degeneration:

Dry form: The “dry” form of macular degeneration is characterized by the presence of yellow deposits, called drusen, in the macula. A few small drusen may not cause changes in vision; however, as they grow in size and increase in number, they may lead to a dimming or distortion of vision that people find most noticeable when they read. In more advanced stages of dry macular degeneration, there is also a thinning of the light-sensitive layer of cells in the macula leading to atrophy, or tissue death. In the atrophic form of dry macular degeneration, patients may have blind spots in the center of their vision. In the advanced stages, patients lose central vision.

Wet form: The “wet” form of macular degeneration is characterized by the growth of abnormal blood vessels from the choroid underneath the macula. This is called choroidal neovascularization. These blood vessels leak blood and fluid into the retina, causing distortion of vision that makes straight lines look wavy, as well as blind spots and loss of central vision. These abnormal blood vessels and their bleeding eventually form a scar, leading to permanent loss of central vision. The dry form of macular degeneration can lead to the wet form. Although only about 10% of people with macular degeneration develop the wet form, they make up the majority of those who experience serious vision loss from the disease.

The data provided by the inventors of the present application show that using a compound of formula I or II in a patient suffering from wet AMD was able to reduce, and in some cases eliminate the extensive fluid formed in the eye (macula).

Thus, in another aspect, the invention provides the a composition comprising at least one Te-containing compound having the formula I or II

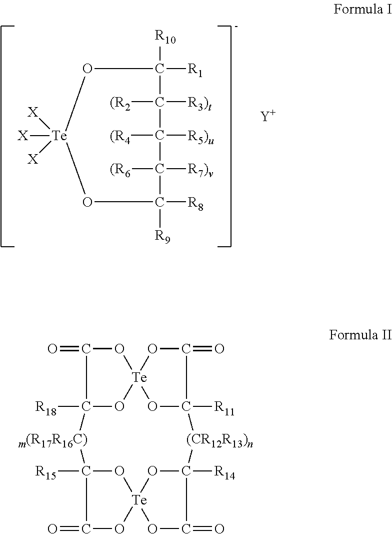

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

for use in the treatment of wet age related macular degeneration and any conditions, diseases, disorders and symptoms thereof in a subject in need thereof.

Thus, in another aspect, the invention provides the a composition comprising at least one Te-containing compound having the formula I or II

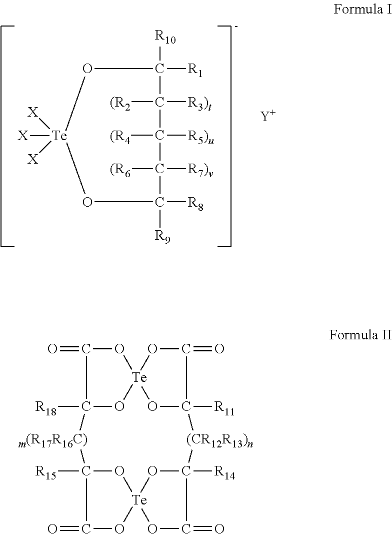

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

for use in the inhibition of the development of wet-AMD in a subject suffering from dry-AMD.

In another aspect the invention provides a method of treating an angiogenesis related condition, disease or disorder comprising administering to a patient in need thereof at least one Te-containing compound selected from

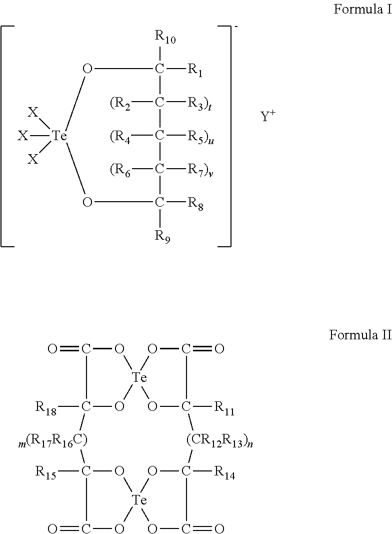

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

wherein said patient has been previously treated with at least one angiogenesis inhibitor (i.e. prior to the administration of said Te-containing compound).

In another aspect the invention provides a method of treating an angiogenesis related condition, disease or disorder (such as for example AMD) comprising administering to a patient in need thereof at least one Te-containing compound selected from

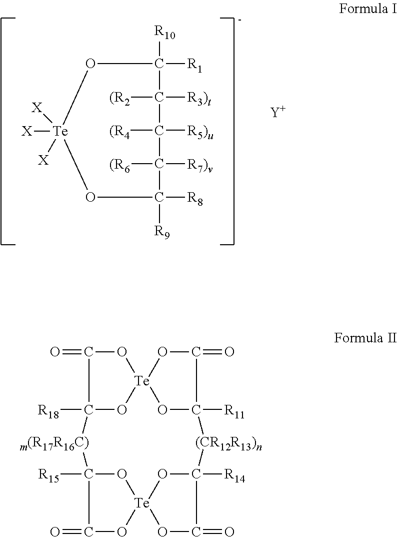

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

wherein said patient is being treated with at least one angiogenesis inhibitor (i.e. during the administration of said Te-containing compound).

The invention further provides the use of at least one Te-containing compound selected from

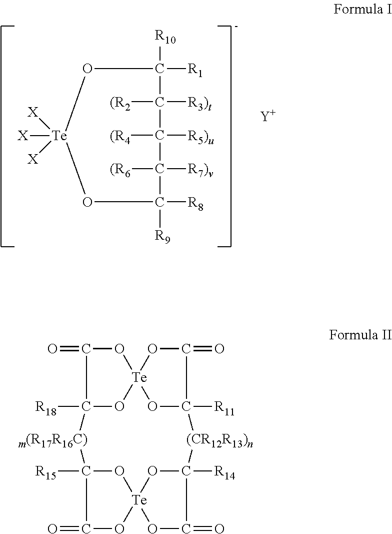

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

in a method of treating an angiogenesis related condition, disease or disorder of a patient in need thereof wherein said patient has been previously treated with at least one angiogenesis inhibitor (i.e. prior to the administration of said Te-containing compound).

The invention also provides the use of at least one Te-containing compound selected from

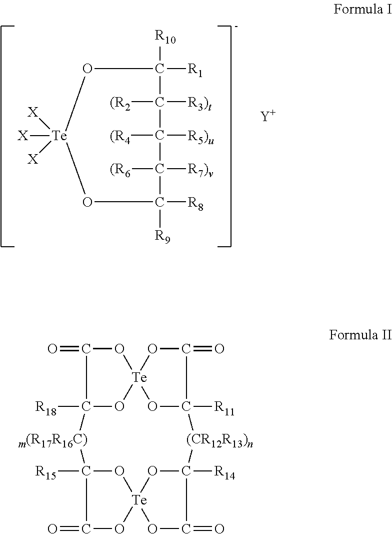

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

in a method of treating an angiogenesis related condition, disease or disorder of a patient in need thereof wherein said patient is being treated with at least one angiogenesis inhibitor (i.e. during the administration of said Te-containing compound).

Typically angiogenesis inhibitors, such as Ranibizumab, are administered chronically to a patient in need thereof (e.g. once a month for an extended period of time). The administration of a Te-containing compound to such patients, reduces the number of times said angiogenesis inhibitor is typically administered. For example, if said patient has been administered with said angiogenesis inhibitor alone every month for the treatment of said angiogenesis condition, disorder or disease, upon administration of a the Te-containing compound said angiogenesis inhibitor will be administered to said patient less frequently than once a month. The administration of said Te-containing compounds increases the time intervals between administration of said angiogenesis inhibitor. If typical time intervals of administration of said angiogenesis inhibitor are once a month, upon administration of said Te-containing compound to such a patient the time interval can increase to at least once every two months, once every 3 months, once every 4 months, once every 5 months, once every 6 months, once every 7 months, once every 8 months, once every 9 months, once every 10 months, once every 11 months, once every 12 months, once every 2 years, once every 3 years. Increasing the time intervals between administrations of angiogenesis inhibitor lowers the risk of side effects related to said agent. For example when angiogenesis inhibitors such as Ranibizumab, which are administered as intra ocular injections every month to a patient suffering from AMD, the administration of a Te-containing compound, such as AS-101, reduces the time intervals between Ranibizumab injections to once every 9 months and lowers the significant side effects associated with such administration of the angiogenesis agent and dramatically increases the patient's quality and standard of living.

In some further aspect the invention provides a method of treating wet-AMD in a subject in need thereof; comprising administering to said subject a composition comprising at least one Te-containing compound having the formula I or II

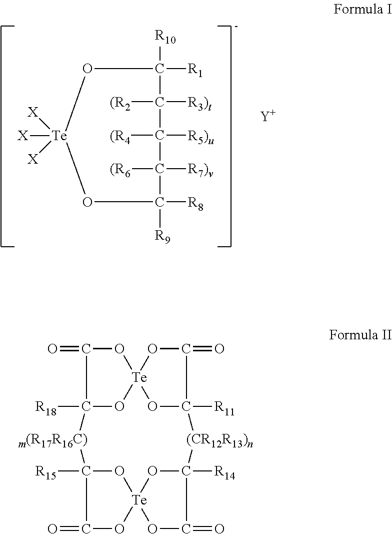

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

for use in the treatment of wet age related macular degeneration and any conditions, diseases, disorders and symptoms thereof in a subject in need thereof.

The invention also includes any salt of a compound of formula I, including any pharmaceutically acceptable salt, wherein a compound of formula I has a net charge and at least one counter ion (having a counter negative or positive charge) is added thereto to form said salt. The phrase “pharmaceutically acceptable salt(s)”, as used herein, means those salts of compounds of the invention that are safe and effective for pharmaceutical use in mammals and that possess the desired biological activity. Pharmaceutically acceptable salts include salts of acidic or basic groups present in compounds of the invention. Pharmaceutically acceptable acid addition salts include, but are not limited to, hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds of the invention can form pharmaceutically acceptable salts with various amino acids. Suitable base salts include, but are not limited to, aluminum, calcium, lithium, magnesium, potassium, sodium, zinc, and diethanolamine salts. For a review on pharmaceutically acceptable salts see BERGE ET AL., 66 J. PHARM. SCI. 1-19 (1977), incorporated herein by reference.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition.

In some embodiments, said subject has low tolerability towards said at least one angiogenesis inhibitor. When referring to low tolerability, it should be understood to include a subject that was previously treated with at least one angiogenesis inhibitor and showed unwanted side effects. In some embodiments said unwanted side effects prevented said subject from continuing to be administered with said at least one angiogenesis inhibitor. Intravitreal anti-VEGF injections are currently the standard of care for AMD and can cause many unwanted side effects such as intraocular pressure, pain, vitreous detachment, intraocular inflammation, dry eye, ocular hyperemia, retinal degeneration, posterior capsule opacification, hemorrhage nasopharyngitis, anemia and more (adverse reactions reported in Lucentis prescribing information in http://www. accessdata.fda.gov/drugsatfda_docs/label/2012/125156s0069s00761bl.pdf).

In some other embodiments, said at least one angiogenesis inhibitor shows low therapeutic effectiveness in the treatment of angiogenesis disease in said subject when administered alone. Thus, under such embodiments, said subject has been previously treated with said at least one angiogenesis inhibitor and showed either no or very slow effective treatment of said disease or loss of treatment efficacy over time. For example in the case of anti-VEGF intravitreal injections there have been reports of efficacy loss after a median of 8 treatments (Retina 2009, June; 29(6): 723-731). A high percentage of non-responders to anti-VEGF treatment in AMD has been reported as well (Br J Ophthalmol 2007, 91: 1318-1322).

In some embodiments, said subject is in need of high dosage of said at least one angiogenesis inhibitor. Thus, under there embodiments the treatment of said angiogenesis disease can be effectively treated by said at least one angiogenesis inhibitor alone, only if given in high dosages or high dosage frequencies.

In further embodiments, said at least one Te-containing compound and at least one angiogenesis inhibitor are administered concomitantly, simultaneously or consecutively.

In some embodiments, said at least one Te-containing compound is administered orally.

In other embodiments, said at least one Te-containing compound is administered daily. In other embodiments, said at least one Te-containing compound is administered at least once a week (i.e. at least once every day, 2, 3, 4, 5, 6, 7 days).

In further embodiments, said at least one angiogenesis inhibitor is administered once a week or less frequently (i.e. in less frequency of administration than once a week, for example once every two weeks and so forth). In some other embodiments, said at least one angiogenesis inhibitor is administered once a month or less frequently (i.e. in less frequency of administration than once a month, for example once every two months and so forth). In some further embodiments, said at least one angiogenesis inhibitor is administered once a year or less frequently (i.e. in less frequency of administration than once a year, for example once every 18 months). In some other embodiments, said at least one angiogenesis inhibitor is administered on an on-demand regimen (i.e. when a medical practitioner assesses there is a medical need for administration of an angiogenesis inhibitor).

The invention further provides the use of at least one Te-containing compound having the formula I or II:

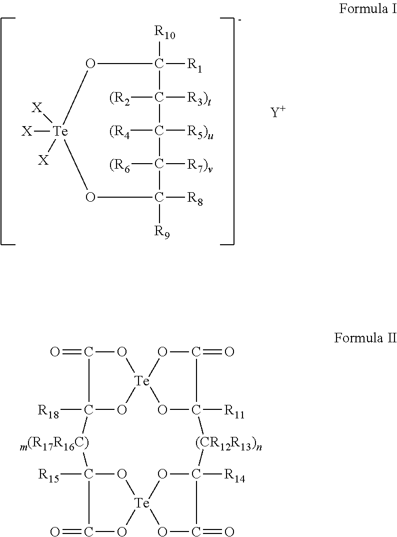

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

for the preparation of a medicament for the treatment of angiogenesis in a patient previously administered with at least one angiogenesis inhibitor.

The invention further provides the use of at least one Te-containing compound having the formula I or II:

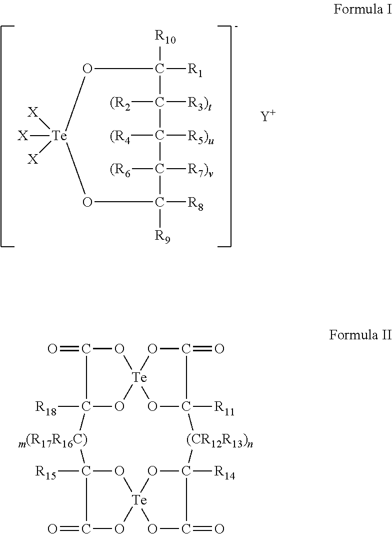

wherein:

each of t, u and v is independently 0 or 1;

each of m and n is independently 0, 1, 2 or 3;

Y is selected from the group consisting of ammonium, phsophonium, potassium, sodium and lithium;

X is a halogen atom; and

each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;

for the preparation of a medicament for the treatment of AMD in a patient previously administered with at least one angiogenesis inhibitor.

When referring to a “patient previously administered with at least one angiogenesis inhibitor” it should be understood to encompass a patient diagnosed with an angiogenesis related disease or disorder (such as for example AMD), which was previous to the administration of said Te-containing compound was administered at least once with at least one angiogenesis inhibitor. In some embodiments said at least one angiogenesis inhibitor is still being administered to said patient. In some embodiments said at least one angiogenesis inhibitor was or is administered once a week or less frequently (i.e. in less frequency of administration than once a week, for example once every two weeks and so forth). In some other embodiments, said at least one angiogenesis inhibitor was or is administered once a month or less frequently (i.e. in less frequency of administration than once a month, for example once every two months and so forth). In some further embodiments, said at least one angiogenesis inhibitor was or is administered once a year or less frequently (i.e. in less frequency of administration than once a year, for example once every 18 months). In some other embodiments, said at least one angiogenesis inhibitor was or is administered on an on-demand regimen (i.e. when a medical practitioner assesses there is a medical need for administration of an angiogenesis inhibitor).

In another aspect, the invention provides a method of treating a subject suffering from AMD (Age related Macular Degeneration), said method comprising the steps of administering to said subject a combination of at least one anti VEGF agent and at least one Te-containing compound of formula I or II.

In some embodiments of a method of the invention the invention, said at least one Te-containing compound is a compound of formula I. In some embodiments, said at least one Te-containing compound is a compound of formula I, wherein t, u and v are each 0 and wherein each of R₁, R₈, R₉ and R₁₀ is hydrogen, wherein X is chloro and wherein Y is ammonium. In some further embodiments, said Te-containing compound is AS-101 below:

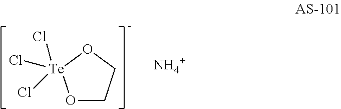

In some other embodiments of a method of the invention, said at least one Te-containing compound is a compound of formula II. In some embodiments, said at least one Te-containing compound is a compound of formula II, wherein each of m and n is 0 and wherein each of R₁₁, R₁₄, R₁₅ and R₁₈ is hydrogen. In some further embodiments, said Te-containing compound is SAS below:

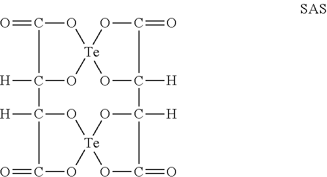

In some embodiments of a method of the invention said subject has been previously treated with anti VEGF agent.

In some other embodiments of a method of the invention, said subject has shown low tolerability or low treatment effect to said anti-VEGF agent.

In further embodiments, said anti VEGF agent is selected from Ranibizumab, Aflibercept, Bevacizumab or any combinations thereof.

In some embodiments of a method of the invention, said at least one Te-containing compound is administered orally.

In some other embodiments of a method of the invention, said at least one Te-containing compound is administered daily to said subject.

In other embodiments of a method of the invention said at least one anti VEGF agent is administered once a week or less.

In some further embodiments of a method of the invention, said at least one anti VEGF agent is administered on an on demand regimen.

Tumor vascular obliteration using combination angiostatic therapy of the invention is associated with reduced tumor mass and increased survival, whereas individual mono-therapies were ineffective. Significant compensatory up-regulation of several pro-angiogenic factors is observed after treatment with a single angiostatic agent. In contrast, treatment with combination angiostatic therapy significantly reduced compensatory up-regulation. Therapies that combine angiostatic molecules targeting multiple, distinct aspects of the angiogenic process represent a previously uncharacterized paradigm for the treatment of many diseases with associated pathological neovascularization.

The exact dose and regimen of administration of the composition will necessarily be dependent upon the therapeutic or nutritional effect to be achieved and may vary with the particular formula, the route of administration, and the age and condition of the individual subject to whom the composition is to be administered.

### DETAILED DESCRIPTION OF EMBODIMENTS

Clinical Study

Patients were orally administered with AS101/Formula I in conjunction with intraocular injections of intra vitreal anti VEGF. None of these two patients responded well to intra vitreal anti VEGF injections alone and were in danger of vision loss. A special compassionate approval has been given by the local committee of ethics in Meir medical center, Kfar Saba, and the Ministry of Health in Israel to treat these two patients with orally taken AS101.

Case 1

The patient was a 63 year old woman was first examined on May 2, 2011, due to metamorphopsia in her right eye. Visual acuity was 20/30 in the right eye and 20/15 in the left eye. The anterior segment and media examinations were unremarkable. Fundus examination revealed large drusen in both eyes. OCT examination showed a juxta foveal PED and sub foveal sub retinal fluid in the right eye, and drusen in the left eye. Patient underwent 3 intra-vitreal Lucentis (Ranibizumab) injections in one month intervals, resulting in the resolution of the subretinal fluid. Four month after the 3rd injection, the patient experienced again metamorphopsia in the right eye and OCT examination showed a new large PED and minimal sub retinal fluid. Patient underwent an additional intra-vitreal Lucentis injection and from this point examination continued every month with treatment in PRN mode. Until Jul. 26, 2012 the patient underwent 9 intra-vitreal Lucentis injections. On January 2013 treatment was switched to Aflibercept (Eylea) and the patient received 3 montly injection and an additional injection on Jun. 18, 2013. During the follow up period, visual acuity ranged between 20/25 and 20/30.

One year after the first injection to the right eye, on May 1, 2012, visual acuity dropped in the left eye to 20/40, subretinal fluid appeared in OCT and the patient began to receive Lucentis injections to the left eye. After the second injection the visual acuity dropped to 20/150 and a large central PED appeared with subretinal fluid over the peak of the PED and over its slope. The patient received an intravitreal triancinolon acetonid injection followed by the 3rd lucentis one week later. A month later, visual acuity improved to 20/25 and the size of the PED decreased significantly, but the subretinal fluid did not resolve. The patient received 2 additional Lucentis injections and on Nov. 27, 2012 treatment was switched to Eylea when it became available in Israel. The patient received 8 monthly Eylea injections, the last one on Jun. 18, 2013. The amount of the subretinal fluid did not change over the peak of the PED as well as at the slope. A posterior sub capsular cataract developed in the left eye and we decide to inject again Lucentis with triamcinilon acetonide prior the cataract surgery.

On Jul. 7, 2013, the patient started to receive 12 drops of AS101 every day orally. On Aug. 13, 2013 the patient received the Lucentis with triamcinilon acetonide injections and OCT one month later showed a decrease of the amount of the subretinal fluid at the base of the PED.

On Sep. 16, 2013 the patient underwent the cataract surgery in the left eye. On the next 3 monthly OCT tests the subretinal fluid at the slope of the PED absorbed for the first time since it appeared and no additional injections were needed in the left eye for 8 months until the discontinuation of the AS101 in Mar. 24, 2014.

In the right eye the patient underwent cataract surgery on Mar. 17, 2014. No intravitreal injections were needed in the right for 10 months, until the discontinuation of the AS101.

In Mar. 24, 2014, AS101 was discontinued. 3 weeks later, on Apr. 13, 2014, OCT test was performed due to new symptom of metamorphopsia showed a development of new subretinal fluid in both eyes.

In another observation found during the compassionate treatment was that drusens/dry AMD did not developed to Wet AMD, suggesting that the treatment of the present application can also prevent or delay the progression of dry AMD to Wet AMD.

Case 2

The patient was a 77 years old man was diagnosed with decreased vision in his left eye. Visual acuity was 20/30 in the right eye and 20/200 in the left eye. Anterior segment and media were normal accept mild nuclear sclerosis in the lens of the right eye and posterior vitreous separation in both eyes. Retina examination revealed in the right eye multiple mid size drusen in the macula, and in the left macula large drusen, central PED and macular hemorrhages. OCT of right eye showed small drusen and in the left eye a central PED and sub retinal fluid at the slope of the PED. Patient received 3 monthly injections of intra vitreal Avastin continued by additional Avastin injections in a PRN mode. When Avastin stopped to respond, treatment was changed to Lucentis injections. During 20 months the patient received 9 Avastin injections and 6 Lucentis injections (total of 15 injections in 20 months). Except for one occasion of a 5 month interval between 2 injections, the patient received an injection every month. His visual acuity ranged between 20/100 and 20/50. Four days after the last Lucentis injection, on 13 Aug. 2013 the patient started to take 12 drops of AS101 orally.

The patient continued to receive Lucentis injections PRN on the following dates: Nov. 8, 2013, Feb. 2, 2014, May 2, 2014, Jul. 3, 2014. On examination on Nov. 11, 2014 there was no need for injection. And in all follow on examinations until January 2016 (no further data is available) The total number of injections during AS101 treatment was 4 in 27 months.

The last visual acuity in Nov. 11, 2014 was 20/30 in the right eye and 20/50 in the left eye. No adverse events were reported in this patient.

TABLE 1
Summary of patients treated with a combination of the invention

| | Angiogenesis | | Angiogenesis |
|---|---|---|---|
| | inhibitor injections | Duration of | inhibitor injections |
| | prior to treatment | treatment with the | during treatment |
| | according to the | combination of the | according to the |
| | invention | invention | invention |
| Patient | RE: 15 injections | 9 months | RE: 0 injections in |
| #1 | (Lucentis or Eylea) | | 9 months |
| | in 20 months | | LE: 1 injection in 9 |
| | LE: 14 injections | | months |
| | (Lucentis or Eylea) | | |
| | in 20 months | | |
| Patient | 15 injections | 27 months Te- | 4 injections in 27 |
| #2 | (Lucentis or | containing | months |
| | Avastin) during 20 | compound: AS 101; | (maximal time |
| | months | Angiogenesis | interval between |
| | | inhibitor: Lucentis) | injections: 14 |
| | | | months) |

We claim:

1. A combination comprising at least one Te-containing compound having the formula I or II: 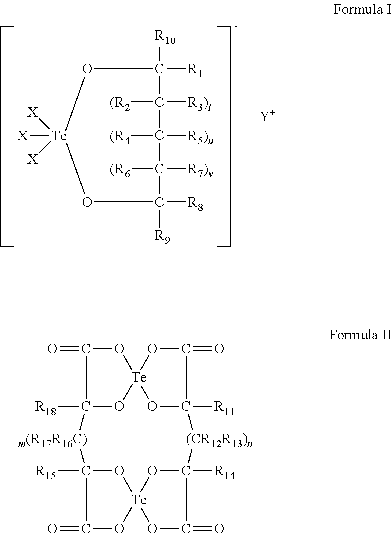
wherein:
each of t, u and v is independently 0 or 1;
each of m and n is independently 0, 1, 2 or 3;
Y is selected from the group consisting of ammonium, phsophonium, potassium, sodium and lithium;
X is a halogen atom; and
each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl; alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;
and at least one angiogenesis inhibitor.

2. A combination according to claim 1, wherein said at least one Te-containing compound is a compound of formula L wherein t, u and v are each 0 and wherein each of R₁, R₈, R₉ and R₁₀ is hydrogen, wherein X is chloro and wherein Y is ammonium.

3. (canceled)

4. A combination according to claim 1, wherein said at least one Te-containing compound is a compound of formula II, wherein each of m and n is 0 and wherein each of R₁₁, R₁₄, R₁₅ and R₁₈ is hydrogen.

5. (canceled)

6. A combination according to claim 1, wherein said at least one angiogenesis inhibitor is selected from Ranibizumab, Aflibercept, Bevacizumab or any combinations thereof.

7. A combination according to claim 1, wherein said at least one Te-containing compound is formulated for oral administration.

8. A combination according to claim 1, wherein said at least one Te-containing compound is formulated for topical administration.

9. A combination according to claim 1, wherein said at least one Te-containing compound is formulated for intraocular administration.

10. A combination according to claim 1, wherein said at least one Te-containing compound is formulated for targeted in-situ administration.

11. A combination according to claim 1, wherein said at least one Te-containing compound is formulated for parenteral administration.

12-24. (canceled)

25. A method of treating an angiogenesis related condition, disease or disorder comprising administering to a patient in need thereof at least one Te-containing compound selected from 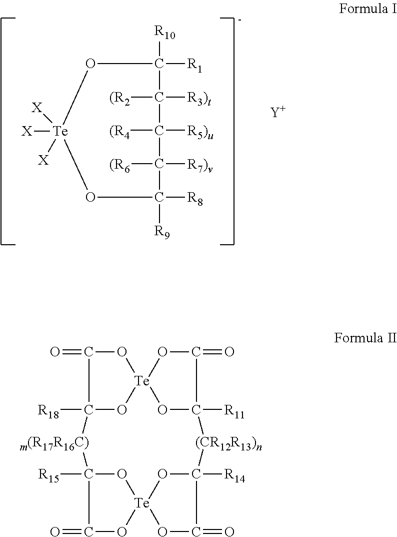
wherein:
each of t, u and v is independently 0 or 1;
each of in and n is independently 0, 1, 2 or 3;
Y is selected from the group consisting of ammonium, phsophonium, potassium, sodium and lithium;
X is a halogen atom; and
each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;
wherein said patient has been previously treated with at least one angiogenesis inhibitor (i.e. prior to the administration of said Te-containing compound).

26. A method of treating an angiogenesis related condition, disease or disorder comprising administering to a patient in need thereof at least one Te-containing compound selected from 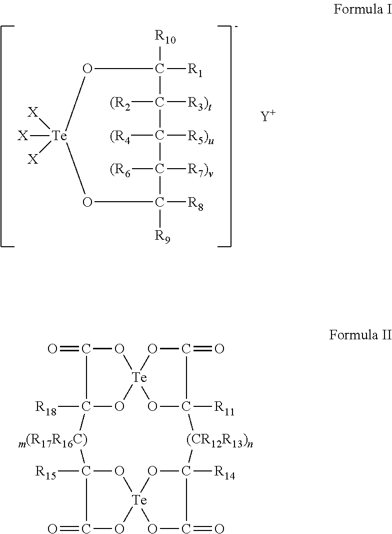
wherein:
each of t, u and v is independently 0 or 1;
each of m and n is independently 0, 1, 2 or 3;
Y is selected from the group consisting of ammonium, phsophonium, potassium, sodium and lithium;
X is a halogen atom; and
each of R₁-R₁₈ is independently selected from the group consisting of hydrogen, hydroxyalkyl, hydroxy, thiohydroxy, alkyl, alkenyl, alkynyl, alkoxy, thioalkoxy, halogen, haloalkyl, carboxy, carbonyl, alkylcarbonylalkyl, carboxyalkyl, acyl, amido, cyano, N-monoalkylamidoalkyl, N,N-dialkylamidoalkyl, cyanoalkyl, alkoxyalkyl, carbamyl, cycloalkyl, heteroalicyclic, sulfonyl, sulfinyl, sulfate, amine, aryl, heteroaryl, phosphate, phosphonate and sulfoneamido;
wherein said patient is being treated with at least one angiogenesis inhibitor (i.e. during the administration of said Te-containing compound).

27. A method of claim 25, wherein said angiogenesis related condition is AMID, said method further comprising the steps of administering to said subject a combination of at least one anti VEGF agent and at least one Te-containing compound of formula I or II.

28-32. (canceled)

33. The method of claim 25, wherein said at least one Te-containing compound is administered orally.

34. The method of claim 25, wherein said at least one Te-containing compound is administered daily to said subject,

35. The method of claim 25, wherein said at least one anti VEGF agent is administered once a month.

36. The method of claim 25, wherein said at least one anti VEGF agent is administered on an on demand regimen.

37-38. (canceled)
